(19) United States
(12) Patent Application Publication
(10) Pub. No.: US 2007/0009583 A1
(43) Pub. Date: Jan. 11, 2007

(54) Dressing

(76) Inventor: Michael H. Qvist, Fredenborg (DK)

Correspondence Address: JACOBSON HOLMAN PLLC, 400 SEVENTH STREET N.W., SUITE 600, WASHINGTON, DC, 20004, US

(21) Appl. No.: 10/575,243

(22) PCT Filed: Oct. 8, 2004
(86) PCT No.: PCT/DK04/00690

§ 371 Date: Apr. 10, 2006

(30) Foreign Application Priority Data
Oct. 10, 2003 (DK) PA2003 05104

(51) Int. Cl. A61K 38/46; A61L 15/00; A61K 9/127

(52) U.S. Cl. 424/445; 424/94.65; 424/450

(57) ABSTRACT

A wound dressing for release of one or more therapeutic ingredients wherein the therapeutic ingredients are contained in liposomes, said liposomes may comprising releasing means being triggered by a wound constituent and thereby releasing the therapeutic ingredients of the liposomes. The dressing further comprises wound exudates handling means and is suitable for moist wound healing.

form of a hydrogel. The absorbent material may preferably be in the form of a layer.

The absorbent layer may have an absorption capacity of 0.9% NaCl aqueous solution at 37° C. of at least 0.05 g/cm², more preferred at least 0.1 g/cm², and most preferred at least 0.2 g/cm², even most preferred at least 0.4 g/cm². In one embodiment of the invention the absorption is at least 0.6 g/cm².

The liposomes used in the present invention may be microscopic spherical vesicles based on small vehicles of lipid bilayers with aqueous environment between the bilayers. Dependent on the constituents used and the production method the liposomes can be made in a wide range in sizes and be either unilamellar (ULV), oligolamellar (OLV) or multilamellar (MLV).

Liposomes have been extensively investigated as drug carriers for skin delivery and parenteral delivery for many drugs. The liposomes form a very stable membrane thus providing a good protection of the active substance and thereby an increased flexibility of the design of the dressing may be achieved. An increased stability of the active ingredient, during production, sterilization and storage may be obtained. Furthermore, liposomes provide an opportunity to incorporate both lipophilic and hydrophilic components without use of any additional agents.

The liposomes may either disrupt slowly without any external mediator or comprise releasing means being triggered by a wound constituent and thereby releasing the therapeutic ingredients of the liposomes.

Lipid components can be cholesterol or various phopholipids, lichitin e.g. phosphatidyl choline, phosphatidyl ethanolamin, phosphatidyl serin and phosphatidyl inositol or mixtures thereof. As lipids may be subjected to oxidation, antioxidants may be added, e.g. tocopherol, butylhydroxitoluen, butylhydroxyianisol, ascorbic acid esters or sodiummetabisulfit.

### BACKGROUND OF THE INVENTION

1. Field of the Invention

The invention relates to wound dressings being capable of releasing one or more therapeutic agents to a wound.

2. Description of the Related Art

Dressings being capable of releasing active agent to a wound are well known in the art. Usually the release from these dressings is dependent on the amount of wound exudate contacting the dressing. This often results in a massive release of active agent in a short period, and not in an amount being adapted to the actual need of the wound.

International patent application No. WO 03/47643 discloses a dressing for wound treatment; the dressing comprises a therapeutic agent and a barrier layer, which separates the therapeutic agent from the wound fluid. The barrier layer comprises a substrate being degradable by specific proteolytic enzymes in the wound fluid. Possible applications of this dressing are limited as the function of the dressing is dependent upon the presence of the specific enzymes.

The difficulties associated with the healing of chronic wounds may be caused by a number of factors, such as arteriosclerosis, heart disease, immune deficits, low blood supply, poor blood perfusion and sometimes poor nutrition status. Typical problems may be bacterial infections, presence of necrotic tissue and/or slough etc. One wound may suffer from more than one of these factors, one end of the wound may have severe necrosis and the other end may suffer from infection. In order to treat such wound more effectively there is a need for a dressing being capable of handling these factors.

However, the therapeutic agents that are used for such wounds are often proteins or other sensible or fragile compounds that are not easily incorporated in a dressing without loss of activity. Thus there is still a need for a wound dressing being able to stabilize such agents during processing and storage and providing a targeted release of the agents to the wound.

### SUMMARY OF THE INVENTION

One object of the invention is to provide a dressing being capable of providing a target release of one or more therapeutic agents to a wound.

Another object of the invention is to stabilize sensible therapeutic agents in wound dressings.

A third object of the present invention is to provide a wound dressing providing an intelligent release of a therapeutical agent.

It has surprisingly been found that by incorporating liposomes into wound dressings a target drug delivery can be achieved by a triggered delivery of therapeutic substances.

### DETAILED DESCRIPTION OF THE PRESENT INVENTION

The invention relates to a wound dressing for targeted release of one or more therapeutic ingredients, wherein the dressing comprises exudates handling means and wherein the therapeutic ingredients are contained in liposomes, said liposomes comprising releasing means being triggered by a wound constituent and thereby releasing the therapeutic ingredients of the liposomes.

The wound dressing of the invention is capable of releasing one or more therapeutic ingredients to a wound, the therapeutic ingredients being contained in liposomes. The wound dressing may be of any suitable construction, such as a foam, matrix, paste or a hydrogel, having wound exudates handling means in the form of absorbent material. The dressing may absorb wound exudate and provide release of active ingredients from the liposomes. The release of active ingredient may be provided by a release of the liposomes from the dressing, or the release may only concern the agent, while the liposomes may stay in the dressing.

The dressing of the present invention may be produced in varying sizes depending on the indication, and in an adhesive version as well as a non-adhesive version. Furthermore, the dressings may be in the form of island dressings, with an adhesive flange surrounding an absorbent element, or the dressing may be in the form of a paste or gel, for cavity filling. Preferably, the dressing of the invention is conformable, soft and flexible.

The dressing may comprise a backing layer, e.g. in the form of a film. This layer may preferably be water impervious but vapor permeable. The layer serves as a barrier against bacteria contamination from the surroundings, and at the same time, the vapor permeability renders it possible for the absorbed moisture (exudates) to evaporate, and thus increase the absorbent capacity of the dressing.

The dressing of the invention comprises wound exudates handling means, thereby providing a moist-wound healing environment.

The dressing may be suitable for any wound especially chronic wounds including leg ulcers, pressure sores, diabetic foot ulcers and burns. The dressing may be used on low to highly exudating wounds. More preferably, the dressing exhibits good retention properties so that the absorbed wound fluid remains in the dressing even when exposed to (some) compression. In this way the surrounding skin may be protected from maceration.

The wound exudates handling means may comprise absorbent material such as hydrocolloids, foam, e.g. polyurethane foam, alginates, chitosan, super absorbent material, e.g. in the form of particles or fibers, fiber material or it may be in the form of a hydrogel. The absorbent material may preferably be in the form of a layer.

The absorbent layer may have an absorption capacity of 0.9% NaCl aqueous solution at 37° C. of at least 0.05 g/cm², more preferred at least 0.1 g/cm², and most preferred at least 0.2 g/cm², even most preferred at least 0.4 g/cm². In one embodiment of the invention the absorption is at least 0.6 g/cm².

The liposomes used in the present invention may be microscopic spherical vesicles based on small vehicles of lipid bilayers with aqueous environment between the bilayers. Dependent on the constituents used and the production method the liposomes can be made in a wide range in sizes and be either unilamellar (ULV), oligolamellar (OLV) or multilamellar (MLV).

Liposomes have been extensively investigated as drug carriers for skin delivery and parenteral delivery for many drugs. The liposomes form a very stable membrane thus providing a good protection of the active substance and thereby an increased flexibility of the design of the dressing may be achieved. An increased stability of the active ingredient, during production, sterilization and storage may be obtained. Furthermore, liposomes provide an opportunity to incorporate both lipophilic and hydrophilic components without use of any additional agents.

The liposomes may either disrupt slowly without any external mediator or comprise releasing means being triggered by a wound constituent and thereby releasing the therapeutic ingredients of the liposomes.

Lipid components can be cholesterol or various phopholipids, lichitin e.g. phosphatidyl choline, phosphatidyl ethanolamin, phosphatidyl serin and phosphatidyl inositol or mixtures thereof. As lipids may be subjected to oxidation, antioxidants may be added, e.g. tocopherol, butylhydroxitoluen, butylhydroxyianisol, ascorbic acid esters or sodiummetabisulfit.

Chronic wounds may be identified by individual indicators present in the wound, e.g. specific proteins or antigens. These may only be present in chronic non-healing wounds and are sometimes a problem in the wound and thereby the target of the therapeutic agent. Sometimes the said indicators are present due to underlying defective condition and are thus not necessarily a problem themselves but may be an indicator of the condition of the wound. Depending on the type of problem related to the wound, the wound constituent may be any biological constituent being present in a chronic wound.

The liposomes may comprise a releasing means, said releasing means may be triggered by a specific wound constituent and the liposomes thereby only releasing the therapeutic agent when exposed to specific wound components. The said wound constituent may only be present in chronic wounds—and may sometimes only be present in specific types of chronic wounds. The wound constituent may be in the wound exudates or bound to the diseased cells in the wound and can be a protein or another type of chemical or biological constituent. The wound constituent may also be a compound produced by microorganisms in the wound.

Liposomes can be custom made such that they are degraded due to the presence of the above-mentioned indicators. The wound constituent may be able to degrade the bilipid layer of the liposomes, or it may trigger means on the surface of the liposomes that induces transport of the therapeutical ingredients over the membrane and thus releasing it to the wound. The wound constituent may interact with or degrade a special component incorporated in the bilayers and thus create a shunt facilitating release of the content of the liposomes, e.g. therapeutic agents. The said agents may have several effects, e.g. a depressing effect on the wound constituent that facilitated the release, thereby creating a self-regulated op-/down-mechanism that determines a window of activity of the wound component or the active agent.

The release may be induced by the action of phospholipases expressed by various bacteria present in the wound. Phospholipases can be divided into four groups depending on the position of the bond they hydrolyse on the phospholipid substrate: phospholipases A1, A2, C and D. Phospholipases C appear to be the most important playing a significant role in bacterial pathogenesis. Phospholipase C (originally called lecithinase and also referred to as a-toxin) catalyzes the hydrolysis of the linkage between glycerol and phosphate in lecithin and other phosphatides. The types of bacteria that commonly cause infection in wounds are Pseudomonas aeruginosa, Staphylococcus aureus and Clostridium perfringens. All of these bacteria have been found to express Phospholipase C. In addition several bacteria (e.g. Pseudomonas aeruginosa) has been shown to produce the phosphatidylcholine (PC)-specific phospholipase D (PLD) that catalyze the hydrolysis of PC to generate choline and phosphatidic acid. A liposome with its content can be prepared such that the release are dependent on degradation of the phospholipid bilayers by bacteria present in a wound.

In one embodiment the therapeutic ingredient may be an enzyme-controlling agent, such as a protease inhibitor. The enzyme may only be present in chronic wound or present in elevated levels compared to healthy human tissue. The release of e.g. a protease inhibitor directly into the wound may ensure a very efficient inhibition of unwanted protease activity. The liposomes may be constructed so that they can diffuse to the cells deep in the wound where the inhibitors are needed. The liposomes may exhibit an intelligent release so that the inhibitor is only released, and in the correct amount, when the protease level is higher than normal. It may be specific proteases that trigger this release. The release may then be up- and down regulated dependent on the level of the said proteases, and thereby normalizing the protease activity level in a window allowing a predetermined activity. E.g. when the dressing is applied to a wound with (too) high protease activity, the proteases facilitate a release from the liposome. The inhibitor is released and will inhibit the protease in the exudate. When most proteases are inhibited no more inhibitor are released until the protease level may rise again.

The release may be obtained by using specific polymers in the liposomes. The released inhibitor may be a synthetic small molecule, such as peptidomimetic or an inhibitor of biological origin, such as antibodies and naturally occurring inhibitors. Examples of such compounds are a-1-antitrypsin (AAT), anti-2-antiplasmin, alpha-2-macroglobulin, Amastatin (HCl), Aminocaproic acid, Anti-chymotrypsin, Anti-trombin, Eglin C, elafin, Epicatechin, EPI-nNE4 (DX-890), Flavonoids (catechin), GM6001, tetracycline, tissue inhibitor of metalloproteinases (TIMP's) or secretory leukocyte protease inhibitor (SLPI).

In one embodiment the therapeutic ingredient may be a tissue healing enhancing agents or growth factor. Growth factors are essential to wound healing because that they are involved in all of the phases of wound healing. Growth factors specifically stimulate the migration and proliferation of cells to the wound and promote the synthesis of new tissue. Since growth factors are proteins and hence sensitive to the production and sterilization methods used when manufacturing dressings, it would be beneficial to incorporate these proteins in liposomes. Examples of such growth factors are platelet derived growth factor (PDGF), epidermal growth factor (EGF), transforming growth factor (TGF), insulin-like growth factor (IGF), fibroblast growth factor (FGF), keratinocyte growth factor (KGF) and vascular endothelial growth factor (VEGF) or RGD tripeptides. It may also be anabolic hormones such as, testosterone or human growth factor (hGH).

The therapeutic ingredient may comprise a pain-reliving agent such as ibuprofen, ketoprofen, flurbiprofen, acetylsalicylic acid, salicylic acid, diclofenac, lornoxicam, indometacin, naproxen, paracetamol (acetaminophen), piroxicam, rofecoxib, tiaprofen acid or tolfenam acid.

In one embodiment the therapeutic ingredient may comprise a bacteriostatic or bactericidal chemical compounds, e.g. iodine, iodopovidone complexes, chloramine, chlorohexidine, silver salts, zinc or salts thereof. Furthermore it may comprise biologic entities such as antibacterial peptides or proteins.

The therapeutic agent may comprise an enzymatic agent. It may be an enzyme promoting wound healing by degrading undesired elements being present in the wound, such as non-viable tissue, non-proliferating cells, wound slough expressed from the immune response of the body due to bacterial activity.

The therapeutic ingredient is a proteolytic enzyme. Proteolytic enzymes are suitable for degrading necrotic tissue and slough and thereby preparing the wound bed for a more effective healing. Examples of suitable proteolytic enzymes are bromelain, collagenase, deoxyribonuclease, fibrinolysin, krillase, papain, pepsin, streptodornase, streptokinase, sutilains, subtilisin, trypsin, vibralysin etc.

In one embodiment the therapeutic ingredient may be DNA or RNA. It is well known that the delivery of DNA or RNA into cells is effectively done using lipid-based formulations in the form of (cationic) liposomes. This is also called gene therapy and is based on the insertion and expression of functional genes or into a living cell, where they are expected to produce a desired therapeutic effect. Usually the liposomes are injected to exert their effect, but by using liposomes with wound triggered release, it could be possible to transfect cells in the wound by applying liposomes topically, released from a wound dressing. The DNA/RNA could be genes encoding for any of the proteins mentioned above (proteases, protease inhibitors, antibacterial peptides or proteins and growth factors) such that the expression of the mentioned peptides/proteins are up- or down-regulated.

An agent having a cooling effect may be comprised in the therapeutic ingredient.

In one embodiment of the invention the dressing of the present invention comprises two or more different therapeutical agents. The different agents may be combined in order to obtain desired properties, e.g. a dressing comprising a debriding enzyme combined with a pain-killing agent. The said agents may be incorporated in liposomes with different release trigger mechanisms, thereby facilitating treatment of a chronic wound where multiple disease patterns are present in either the same part of the wound or in different local areas of the wound. Using the one and same dressing several therapeutic goals can be reached using liposomes with different trigger-mechanisms. E.g. one part of the wound can be treated for to high protease levels, whereas another part of the wound can be treated with growth factors etc.

### EXAMPLES

### Example 1A

### Preparation of (MLV) Liposomes

Liposomes were prepared using the solvent evaporation technique. 5 ml of an egg yolk emulsion 30% (vol/vol), obtained from Sigma-Aldrich was transferred to a round-bottomed flask and added 20 ml of methanol. Water and methanol was evaporated under reduced pressure at 65° C. for 15 min., using the rotavapor. The lipid (approx. 1.0 ml) was diluted in 10 ml of chloroform/methanol. 5 ml of this solution was dried, under nitrogen gas and 20 ml of a 5% w/vol papain PBS (phosphate buffered saline) solution (pH 7.4) was added. The solution was kept in a closed container under nitrogen and vortexed for 30 min. thereafter it was frozen to −80° C. and after 5 min thawed to at 45° C. This was repeated 3 times. Using flow-through dialysis with a cellulose ester dialysis membrane, with cut off at 50 kDa (Spectra/Por® MacroDialyzers from Spectrum Laboratories, Inc, CA, USA) the untrapped papain and other lipid components were removed. Washing was performed 3 times with PBS-buffer solution (pH 7.4). Multilamellar liposomes were obtained with a mean diameter of 2 μm, measured by light scattering measurement and the papain content was measured to 12% (w/w) (BCA assay) and an specific activity to 1×10⁵ USP units pr gr. (BAPA assay). The liposomes (100 mg/ml) were kept in PBS buffer at 25° C. No significant change in stability was observed for 3 months.

### Example 1B

### Preparation of (ULV) Liposomes

Liposomes were prepared as in Example 1A. However, after the freeze and thaw process the solution was ultrasonificated at 60° C. at 45 min. Flow-through dialysis and washing was performed as in Example 1A. Unilamellar liposomes were obtained with a mean diameter of 100 nm, and a papain content of 2% (w/w). The liposomes (100 mg/ml) were kept in PBS buffer at 25° C. No significant change in stability was observed for 3 months.

### Example 2A

### Foam Dressing with Liposomes Incorporated

The liposomes prepared in Example 1A and 1B were incorporated into a foam wound dressing by mixing 2 ml liposome solution, 8 g Hypol2060 (Dow Chemical Company), 12 g of Hypol 2002 and 18 g of water with 1% w/w Pluronic 62 (BASF). The materials were mixed together for approximately 15 seconds. The liquid was poured into a mould and allowed to react for 10 minutes. The resulting foam sheet was dried at reduced pressure at room temperature for 24 hours. The foam had a thickness of 3 mm and a polyurethane (PU) backing film was laminated on the top of the foam thus sealing the dressing from outside. Using Franz diffusion chambers (Permgear), a release assay in saline phosphate buffer (pH 7.4) was performed. Protein determination (BCA assay) and activity (BAPA assay) were made. For both preparations, 95% of the enzyme was released within 96 hours and no significant loss in activity was observed.

### Example 2B

### Foam Dressing with Liposomes in a Film

1 ml of the liposome solutions prepared in Example 1A and 1B were added to 9 ml of a PVP K90 solution (10%, w/vol) in ethanol/water (50:50, vol/vol). The solution was casted onto a release liner and the solvent allowed to evaporate, thereby obtaining a film of 100 gsm (gr/m²). The release liner with film was applied to a foam prepared as in example 2A (but wit no liposomes). Release assay, protein content and activity was measured as in example 2A. For both preparations, 95% of the enzyme was released within 24 hours and no significant loss in activity was observed.

### Example 2C

### Foam Dressing with Liposomes Incorporated and in a Film

A film as prepared in Example 2B was applied to a foam with liposomes as prepared in Example 2A. Release assay, protein content and activity was measured as in Example 2A. For both preparations an initial burst release was observed, where approximately 33% of total enzyme was release within 24 hours, followed by a slower release rate. After 96 hours approximately 95% of the enzyme was released and no significant loss in activity was measured.

### Example 2D

### Hydrogel Dressing with Liposomes

Liposomes were prepared as in Example 1A and 1B. The liposomes were gently suspended into a hydrogel (ratio 1:10) corresponding to European Patent No. 928 206. Using Munktell paper membranes (ooM, Grycksbo) a release assay in Franz diffusion chambers was performed. Protein content and activity was measured as in Example 2A. For both preparations, 95% of the enzyme was released within 36 hours and no significant loss in activity was measured.

### Example 3A

### Preparation of Liposomes with Release Trigged by Serum/Exudate

A hydrogel with liposomes was prepared as in Example 2D. A sandwich-ELISA assay was prepared. Release of papain from the liposomes was measured using antibodies for papain (Goat anti-PAPAIN from Research Diagnostics Inc). This was done in two different release media, an ordinary saline phosphate buffer (pH 7.4) and in non-deactivated calf serum (from Sigma Aldrich). The results showed that the release rate was approximate twice as high in calf serum than in the phosphate buffer. This indicated that constituents of the serum facilitate release from the liposomes. Wound exudates have to some extent similar properties and constituents as serum and may be used as a substitute for wound exudates in in-vitro assays, and may in this Example illustrate the impact of the exudates to the liposomes.

### Example 3B

### Preparation of Liposomes with Targeting of Protease Activity

A hydrogel with liposomes were prepared as in Example 2D. Liposomes were prepared as in Example 1A, however instead of papain, the inhibitor 1AA was used and to the egg yolk emulsion was added collagen (Collagen calf skin, Type I, solution 1 mg/ml from Sigma-Aldrich). A release assay was performed. The release media was added protease collagenase, MMP VIII. A sandwich-Elisa assay indicated that, approximately 95% of the enzyme was released within 12 hours.

### Example 3C

### Preparation of Liposomes with Targeting of Bacterial Phopholipase Activity

A hydrogel with liposomes was prepared as in Example 2D and a release assay was performed. The release media was added bacteria (1% Pseudomonas aeruginosa in suspension). The activity measurement indicated that for both preparations, approximately 95% of the enzyme was released within 12 hours.

1. A wound dressing for targeted release of one or more therapeutic ingredients, wherein the dressing comprises exudates handling means and wherein the therapeutic ingredients are contained in liposomes, said liposomes comprising releasing means being triggered by a wound constituent and thereby releasing the therapeutic ingredients of the liposomes.

2. A wound dressing according to claim 1, wherein the therapeutic ingredient comprises a protease-controlling agent.

3. A wound dressing according to claim 1, wherein the therapeutic ingredient comprises a growth factor, growth hormone or other chemical/biological agent facilitating growth and healing of wounds.

4. A wound dressing according to claim 1, wherein the therapeutic ingredient comprises an antibacterial agent.

5. A wound dressing according to claim 1, wherein the therapeutic ingredient comprises a pain-relieving agent.

6. A wound dressing according to claim 1, wherein the therapeutic ingredient comprises an enzymatic agent.

7. A wound dressing according to claim 1, wherein the therapeutic ingredient comprises a proteolytic enzyme.

8. A wound dressing according to claim 1, wherein the proteolytic enzyme comprises papain.

9. A wound dressing according to claim 1, wherein the wound constituent is a biological constituent being present in a chronic wound.

10. A wound dressing according to claim 9, wherein the wound constituent is present in the wound exudates.
